(19) United States
(12) Patent Application Publication
(10) Pub. No.: US 2016/0106963 A1
(43) Pub. Date: Apr. 21, 2016

(54) Hydration maintenance slipper

(71) Applicants: Susan Mowers, Richland, NY (US); Irma Bush, Richland, NY (US)

(72) Inventors: Susan Mowers, Richland, NY (US); Irma Bush, Richland, NY (US)

(21) Appl. No.: 14/887,293

(22) Filed: Oct. 19, 2015

Related U.S. Application Data
(60) Provisional application No. 62/065,078, filed on Oct. 17, 2014.

(51) Int. Cl. A61M 35/00; A43B 7/00; A43B 3/10

(52) U.S. Cl. CPC A61M 35/00; A43B 3/106; A43B 3/107; A43B 7/00

(57) ABSTRACT

A hydration maintenance slipper maintains moisture in the user's feet while they sleep. The slipper may be reusable or disposable. An internal pad is saturated with a skin lotion or other treatment of the user's choice. The slipper may be comfortably worn around the house, and the disposable version is thin enough to be worn inside conventional boots or shoes.

### CROSS-REFERENCE TO RELATED APPLICATIONS

This Application claims the benefit of U.S. Provisional Application No. 62/065,078, filed Oct. 17, 2014, which is hereby incorporated by reference.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to foot care equipment and accessories and in particular to a hydration maintenance slipper. For many people, cracked and dry skin on the soles of the feet can be an unsightly and painful problem. It prevents them from participating in certain outdoor activities such as beach excursions, and makes them reluctant to wear certain types of shoes and sandals which expose the feet to the view of others. Frequent pedicures are an unsatisfactory solution since they are time consuming and expensive. Keeping the skin on the soles of the feet moistened and hydrated would prevent this problem from occurring. A hydration maintenance slipper, which provides an internal pad saturated with a skin lotion or other treatment of the user's choice and may be comfortably worn around the house, would resolve this problem.

### SUMMARY OF THE INVENTION

Accordingly, the invention is directed to a hydration maintenance slipper. The slipper may be reusable or disposable. An internal pad is saturated with a skin lotion or other treatment of the user's choice. The slipper may be comfortably worn around the house, and the disposable version is thin enough to be worn inside conventional boots or shoes.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWINGS

The accompanying drawings are included to provide a further understanding of the invention and are incorporated into and constitute a part of the specification. They illustrate two embodiments of the invention and, together with the description, serve to explain the principles of the invention.

FIG. 1 is a side perspective view of the first exemplary embodiment worn on the user's feet, displaying the disposable slippers 10, and the cuffs 10A.

FIG. 2 is a side view of the first exemplary embodiment, displaying the disposable slippers 10, and the cuffs 10A.

FIG. 3 is a top perspective view of the second exemplary embodiment, displaying the reusable slippers 11, and the pads 12.

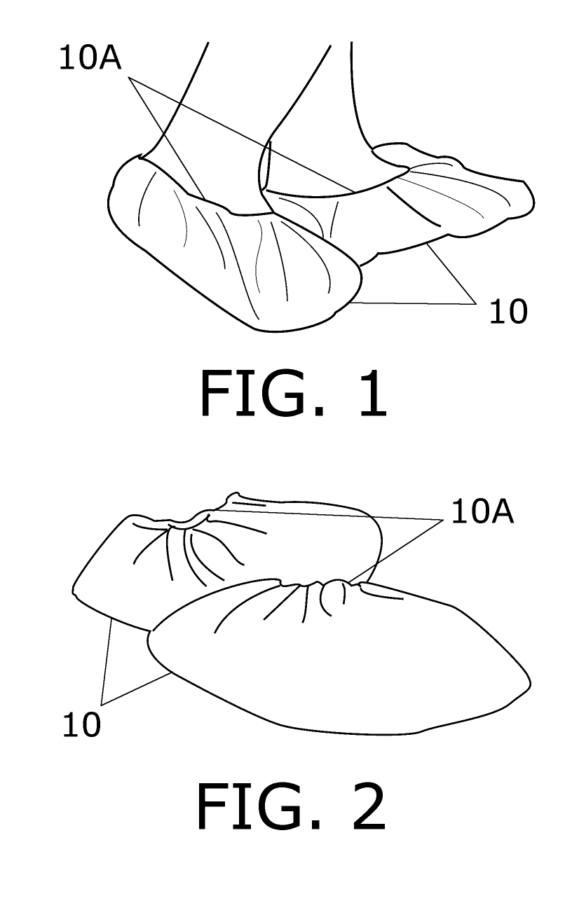

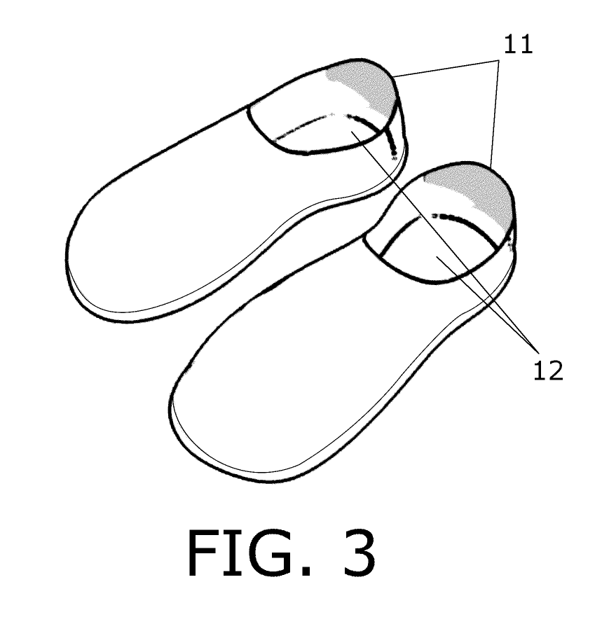

### DETAILED DESCRIPTION OF THE INVENTION

Referring now to the invention in more detail, the invention is directed to a hydration maintenance slipper. An internal pad 12 is saturated with a skin lotion or other treatment of the user's choice.

The first exemplary embodiment provides a disposable slipper 10, preferably manufactured from a flexible, disposable material such as paper, plastic or latex. The disposable slipper 10 is preferably secured to the user's foot with an elastic ankle cuff 10A. Preferably, the pad 12 is permanently adhered to the interior of the disposable slipper 10. A large plurality of disposable slippers 10, such as 50 disposable slippers 10, may be provided in a box or other package, in the same manner as facial tissues or latex gloves. The disposable slippers 10 are thin enough to be worn inside conventional boots or shoes.

To use the first exemplary embodiment, the user may remove a pair of disposable slippers 10 and put them on the feet.

The second exemplary embodiment provides a reusable slipper 11, which is preferably manufactured from a flexible, durable material such as silicone, plastic, or rubber. Preferably, the pad 12 is packaged separately and is disposable. The pad 12 may be adhered to the interior surface of the reusable slipper 11 with peel-and-stick adhesive strips on the bottom surface of the pad 12.

To use the second exemplary embodiment, the user may place a pad 12 in each reusable slipper 11, and wear the reusable slippers.

Both the first and second exemplary embodiments may be comfortably worn around the house. The pad 12 is preferably provided in a fully prepared condition for immediate use, containing an ointment such as skin lotion or moisturizer within the fabric of the pad. Medicated pads 12 may be provided for users with particular medical conditions, such as diabetes, toenail fungus, or athlete's foot. Alternate embodiments, wherein the pad 12 may be provided dry, such that the user may apply a skin lotion, moisturizer, or other product of the user's choice, are contemplated.

The disposable slipper 10 is preferably manufactured from a flexible, disposable material such as paper, plastic or latex. The cuff 10A is preferably manufactured from a flexible, durable fabric which is elastic, such as elasticized cotton. The reusable slipper 11, which is preferably manufactured from a flexible, durable material such as silicone, plastic, or rubber. The pad 12 is preferably manufactured from a flexible, disposable material which is absorbent, such as cotton gauze.

Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. A hydration maintenance slipper, comprising:
(a) a slipper having an interior;
(b) an internal pad;
(c) said internal pad being attached to the interior of said slipper;
(d) said internal pad being saturated with an ointment.

2) The hydration maintenance slipper of claim 1, wherein said slipper is disposable; said slipper being made of a disposable material such as paper, plastic, or latex.

3) The hydration maintenance slipper of claim 2, further comprising an elastic ankle cuff; said elastic ankle cuff being configured to secure said slipper onto a foot.

4) The hydration maintenance slipper of claim 2, wherein said internal pad is permanently adhered to the interior of said slipper.

5) The hydration maintenance slipper of claim 3, wherein said internal pad is permanently adhered to the interior of said slipper.

6) The hydration maintenance slipper of claim 2, wherein said disposable material is thin enough to be worn inside conventional boots or shoes.

7) The hydration maintenance slipper of claim 3, wherein said disposable material is thin enough to be worn inside conventional boots or shoes.

8) The hydration maintenance slipper of claim 1, wherein said slipper is reusable; said slipper being made of a flexible durable material such as silicone, plastic, or rubber.

9) The hydration maintenance slipper of claim 8, wherein said internal pad is removably attached to the interior of said slipper and is replaceable.

10) The hydration maintenance slipper of claim 9, wherein said internal pad is removably attached to the interior of said slipper by means of peel-and-stick adhesive strips on the bottom of said internal pad.

11) The hydration maintenance slipper of claim 1, wherein said ointment is any one of the following group: lotion, moisturizer, medication, creams, or gels.

12) The hydration maintenance slipper of claim 2, wherein said ointment is any one of the following group: lotion, moisturizer, medication, creams, or gels.

13) The hydration maintenance slipper of claim 3, wherein said ointment is any one of the following group: lotion, moisturizer, medication, creams, or gels.

14) The hydration maintenance slipper of claim 4, wherein said ointment is any one of the following group: lotion, moisturizer, medication, creams, or gels.

15) The hydration maintenance slipper of claim 5, wherein said ointment is any one of the following group: lotion, moisturizer, medication, creams, or gels.

16) The hydration maintenance slipper of claim 6, wherein said ointment is any one of the following group: lotion, moisturizer, medication, creams, or gels.

17) The hydration maintenance slipper of claim 7, wherein said ointment is any one of the following group: lotion, moisturizer, medication, creams, or gels.

18) The hydration maintenance slipper of claim 8, wherein said ointment is any one of the following group: lotion, moisturizer, medication, creams, or gels.

19) The hydration maintenance slipper of claim 9, wherein said ointment is any one of the following group: lotion, moisturizer, medication, creams, or gels.

20) The hydration maintenance slipper of claim 10, wherein said ointment is any one of the following group: lotion, moisturizer, medication, creams, or gels.
